(19) United States
(12) Patent Application Publication
(10) Pub. No.: US 2007/0032456 A1
(43) Pub. Date: Feb. 8, 2007

(54) Modulation of cell death

(76) Inventor: Albert David Friesen, Winnipeg (CA)

Correspondence Address: MERCHANT & GOULD PC, P.O. BOX 2903, MINNEAPOLIS, MN, 55402-0903, US

(21) Appl. No.: 10/551,302

(22) PCT Filed: Mar. 25, 2004
(86) PCT No.: PCT/IB04/00899

§ 371 Date: Jul. 20, 2006

Related U.S. Application Data
(60) Provisional application No. 60/458,167, filed on Mar. 27, 2003.

(51) Int. Cl. A61K 31/675; A61K 31/4415

(52) U.S. Cl. 514/89; 514/350

(57) ABSTRACT

A method of modulating cell death includes administering pyridoxal-5′phosphate, pyridoxal, pyridoxine, pyridoxamine, 3-acylated analogues of pyridoxal, 3-acylated analogues of pyridoxal-4,5-aminal, pyridoxine phosphonate analogues, or pharmaceutical compositions thereof.

This application claims priority to U.S. Provisional Application No. 60/458,167 filed on Mar. 27,2003 entitled MODULATION OF CELL DEATH the disclosure of which is incorporated by reference herein.

### BACKGROUND

Cell death may arise through a variety of mechanisms. Several of these mechanisms are well characterized including apoptosis and necrosis.

Apoptosis, also known as programmed cell death, can be distinguished from necrosis by a variety of characteristics. During apoptosis, an ATP dependent process, the cellular DNA breaks down into specific sized 185 base pair fragments; the cells shrink; specific cellular proteins (such as caspases) are activated; and the cellular membrane remains intact while blebbing and producing apoptotic bodies.

Conversely, necrosis is characterized by randomly sized DNA fragments, free radical formation, swelling of the cell, and loss of membrane integrity resulting in the release of cellular contents.

Cell death has been implicated in a number of disease states. Cell death can also result from traumatic injuries due to cellular damage from the mechanical stress and the inflammatory response. Because of the influence of cell death in some disease states, and traumatic injuries, there remains a need for methods of modulating cell death.

### SUMMARY OF THE INVENTION

The invention is directed to, a method of modulating cell death that includes administering a therapeutically effective amount of at least one of pyridoxal-5′-phosphate, pyridoxal, pyridoxic acid, pyridoxine, pyridoxamine, 3-acylated analogues of pyridoxal, 3-acylated analogues of pyridoxal-4,5-aminal, pyridoxine phosphonate analogues, or pharmaceutical compositions thereof.

### BRIEF DESCRIPTION OF THE DRAWINGS

FIG. 1 depicts levels of IL-6 produced in cells treated with 0, 50, 100, 250, 500, and 1000 nM pyridoxal-5′-phosphate respectively.

FIG. 2 depicts levels of IL-6 in cells treated with 100 nM pyridoxal-5′-phosphate 0, 2, 4, 6 and 12 hours after application of oxidative stress.

### DESCRIPTION OF THE INVENTION

The recitation of numerical ranges by endpoints includes all numbers and fractions subsumed within that range (e.g. 1 to 5 includes 1, 1.5, 2, 2.75, 3, 3.80, 4, and 5 for example).

All numbers and fractions thereof are presumed to be modified by the term “about.”

It is to be understood that “a,” “an,” and “the” include plural referents unless the content clearly dictates otherwise. Thus, for example, reference to a composition containing “a compound” includes a mixture of two or more compounds.

Some of the compounds described herein contain one or more asymmetric centers and may thus give rise to enantiomers, diastereomers, and other stereoisomeric forms which may be defined in terms of absolute stereochemistry as (R)— or (S)—. The present invention is meant to include all such possible diastereomers and enantiomers as well as their racemic and optically pure forms. Optically active (R)— and (S)— isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise all tautomeric forms are intended to be included.

The invention is directed to methods of modulating cell death by administering pyridoxal-5′-phosphate (also referred to herein as either PLP or P5P), pyridoxal, pyridoxic acid, pyridoxine, pyridoxamine, 3-acylated analogues of pyridoxal, 3-acylated analogues of pyridoxal-4,5-aminal, pyridoxine phosphonate analogues, pharmaceutically acceptable salts thereof, or a pharmaceutical composition thereof.

As used herein, the phrase “modulating cell death” includes but is not limited to, preventing the death of at least one cell, decreasing the rate at which at least one cell dies, decreasing the number of cells that die due to a disease state or traumatic injury, and/or decreasing or modifying cellular stress or dysfunction that may lead or contribute to cell death.

Pyridoxal-5′-phosphate, pyridoxal, pyridoxine, pyridoxic acid, pyridoxamine, 3-acylated analogues of pyridoxal, 3-acylated analogues of pyridoxal-4,5-aminal, pyridoxine phosphonate analogues, pharmaceutically acceptable salts thereof, or pharmaceutical compositions thereof can be used in methods of modulating cell death.

For methods of the invention, a therapeutic compound including any one or more of pyridoxal-5′-phosphate, pyridoxal, pyridoxic acid, pyridoxine, pyridoxamine, 3-acylated analogues of pyridoxal, 3-acylated analogues of pyridoxal-4,5-aminal, pyridoxine phosphonate analogues, pharmaceutically acceptable salts thereof, or pharmaceutical compositions thereof can be administered in a therapeutically effective amount to a patient.

A “therapeutically effective amount” as used herein includes a prophylactic amount, for example, an amount effective for preventing the death of at least one cell. A therapeutically effective amount also includes an amount effective for decreasing the rate at which at least one cell dies. A therapeutically effective amount also includes an amount effective for decreasing the number of cells that die due to a disease state or traumatic injury. A therapeutically effective amount also includes an amount effective for decreasing or modifying cellular stress or dysfunction that may lead to or contribute to cell death.

A therapeutic compound can be administered, for example, after a disease state in which cellular death plays a role, has been diagnosed. In an alternative embodiment, a composition of the invention can be administered after a traumatic injury that is likely to cause cell death. A therapeutic compound can also be administered before the onset of an event or disease state in which cellular death plays a role.

Cell death has been implicated in a number of disease states. As an example, oxidative stress can cause cell death and can arise from disease states such as diabetes, pancreatitis, liver damage, leaky gut syndrome, Parkinson's disease, Alzheimer's disease, Multiple Sclerosis, artherosclerosis, intermittent claudication, peripheral vascular disease, asthma, emphysema, chronic pulmonary disease cataracts, retinopathy, macular degeneration, rheumatoid arthritis, glomerulonephritis, age spots, vitiligo, wrinkles, accelerated aging, cancer, autoimmune diseases, sepsis, inflammatory states, AIDS, and Lupus for example.

Cell death can also result from traumatic injuries due to cellular damage from mechanical stress, damage precipitating from surgical trauma or physical injury, and the inflammatory response for example. Examples of inflammatory disorders are those where inflammation plays a pathogenetic role, include but not limited to Alzheimer's disease, anaphylaxis, ankylosing spondylitis, asthma, atopic dermatitis, chronic obstructive pulmonary disease, Crohn's disease, gout, Hashimoto's thyoiditis, Multiple Sclerosis, osteoarthritis, pemphigus, periodic fever syndromes, psoriasis, rheumatoid arthritis, sarcoidosis, systemic lupus erythematosis, ulcerative colitis, vasculitides (Werner's syndrome, Goodpasture's syndrome, giant cell arteritis, polyareritis nodosa), and xenograft rejection for example. Inflammatory disorders of infectious origin include but are not limited to bacterial dysentery, Chagas disease, cystic fibrosis pneumonitis, filariasis, Helicobacter pylori gastritis, Hepatitis C, influenza virus pneumonia, Leprosy (tuberculoid form) Neisserial or pneumococcal meningitis, post-streptococcal glomerulonephritis, Sepsis syndrome, and Tuberculosis. Inflammatory diseases causing post-inflammatory fibrosis include Bleomycin-induced pulmonary fibrosis, Chronic allograft rejection, idiopathic pulmonary fibrosis, hepatic cirrhosis (post-viral or alcoholic), radiation-induced pulmonary fibrosis, and Schistosomiasis. (Carl Nathan, “Points of Control in Inflammation”, Nature, vol 420, December 2002)

IL-6, a cytokine, has been shown to be a key mediator of inflammation. It has also been shown to both promote and suppress cell proliferation. IL-6 promotes the growth of human myeloma cells and when the IL-6 function is blocked the growth is inhibited. IL6 blocks the growth of some solid tumors such as mammary carcinomas, cervical carcinomas, human lung cancer cell lines, histiocytic lymphomas, and melanomas. Control of key players involved with inflammation, cell death, and cell survival may lead to the ability to dramatically alter associated disease states. Therefore, another embodiment of the invention includes a method of moderating IL-6.

Therapeutic Compounds Suitable for Use in Methods of the Invention

Methods of the invention include administration of a therapeutically effective amount of a compound including any one or more of pyridoxal-5′-phosphate, pyridoxal, pyridoxine, pyridoxamine, 3-acylated analogues of phosphate analogues, pharmaceutically acceptable salts thereof, or pharmaceutical compositions thereof.

In one embodiment, a therapeutic compound includes any one or more of pyridoxal-5′-phosphate, pyridoxic acid, pyridoxal, pyridoxine, pyridoxamine, or a pharmaceutically acceptable salt thereof.

Pyridoxal-5′-phosphate (PLP), an end product of vitamin B₆ metabolism, plays a vital role in mammalian health. Vitamin B₆ typically refers to pyridoxine, which is chemically known as 2-methyl-3-hydroxy-4,5-di(hydroxymethyl)pyridine and is represented by formula I: 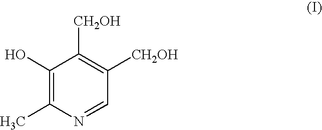
Yet two additional compounds, pyridoxal (formula II) 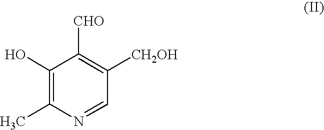
and pyridoxamine (formula III) 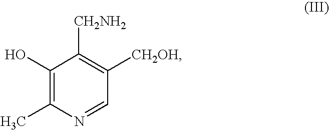
are also referred to as vitamin B₆. All three compounds serve as precursors to pyridoxal-5′-phosphate (PLP), which is chemically known as 3-hydroxy-2-methyl-5-[(phosphonooxy) methyl]-4-pyridinecarboxaldehyde and is represented by formula IV:

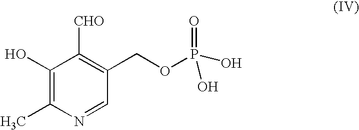

PLP is the biologically active form of vitamin B₆ inside cells and in blood plasma. Mammals cannot synthesize PLP de novo and must rely on dietary sources of precursors such as pyridoxine, pyridoxal, or pyridoxamine, which are metabolized to PLP. For instance, mammals produce PLP by phosphorylating pyridoxine by action of pyridoxine kinase and then oxidizing the phosphorylated product to form PLP.

PLP is a regulator of biological processes and a cofactor in more than 100 enzymatic reactions. It has been shown to be an antagonist of a purinergic receptor, thereby affecting ATP binding; it has been implicated in modulation of platelet aggregation; it is an inhibitor of certain phosphatase enzymes; and it has been implicated in the control of gene transcription. PLP is also a coenzyme in certain enzyme-catalyzed processes, for example, in glycogenolysis at the glycogen phosphorylase level, in the malate asparatate shuttle involving glycolysis and glycogenolysis at the transamination level, and in homocysteine metabolism. In previous patents (U.S. Pat. No. 6,051,587 and U.S. Pat. No. 6,043,259 which are incorporated by reference herein) the role of pyridoxal-5′-phosphate, and its precursors pyridoxal and pyridoxine (vitamin B₆), in mediating cardiovascular health and in treating cardiovascular related diseases has been disclosed.

Therapeutic compounds include esters of pyridoxic acid and pyridoxic acid4,5-lactone.

Therapeutic compounds also include any one or more of the 3-acylated analogues of pyridoxal represented by formula V: 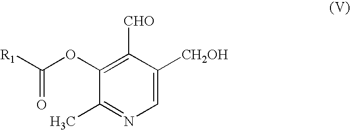
where
R₁ is alkyl or alkenyl, in which alkyl or alkenyl can be interrupted by nitrogen, oxygen, or sulfur, and can be unsubstituted or substituted at the terminal carbon with hydroxy, alkoxy, alkanoyloxy, alkanoyloxyaryl, alkoxyalkanoyl, alkoxycarbonyl; R₁ is dialkylcarbamoyloxy, alkoxy, dialkylamino; alkanoyloxy, alkanoyloxyaryl; alkoxyalkanoyl; alkoxycarbonyl; dialkylcarbamoyloxy; R₁ is aryl, aryloxy, arylthio, or aralkyl, in which aryl can be substituted by alkyl, alkoxy, amino, hydroxy, halo, nitro, or alkanoyloxy,

or a pharmaceutically acceptable salt thereof.

The term “alkyl” includes a straight or branched saturated aliphatic hydrocarbon radicals, such as, for example, methyl, ethyl, propyl, isopropyl (1-methylethyl), 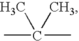
butyl, tert-butyl (1,1-dimethylethyl), and the like.

The term “alkenyl” includes an unsaturated aliphatic hydrocarbon chain having from 2 to 8 carbon atoms, such as, for example, ethenyl, 1-propenyl, 2-propenyl, 1-butenyl, 2-methyl-1-propenyl, and the like.

The above alkyl or alkenyl can optionally be interrupted in the chain by a heteroatom, such as, for example, a nitrogen, sulfur, or oxygen atom, forming an alkylaminoalkyl, alkylthioalkyl, or alkoxyalkyl, for example, methylaminoethyl, ethylthiopropyl, methoxymethyl, and the like.

The above alkyl or alkenyl can optionally be substituted at the terminal carbon by hydroxy, alkoxy, alkanoyloxyaryl, alkanoyloxy, alkoxyalkanoyl, alkoxycarbonyl, or dialkylcarbamoyloxy.

The term “alkoxy” (i.e. alkyl-O—) includes alkyl as defined above joined to an oxygen atom having preferably from 1 to 4 carbon atoms in a straight or branched chain, such as, for example, methoxy, ethoxy, propoxy, isopropoxy (1-methylethoxy), butoxy, tert-butoxy (1,1-dimethylethoxy), and the like.

The term “dialkylamino” includes two alkyl groups as defined above joined to a nitrogen atom, in which alkyl has preferably 1 to 4 carbon atoms, such as, for example, dimethylamino, diethylamino, methylethylamino, methylpropylamino, diethylamino, and the like.

The term “alkanoyloxy” includes a group of the formula 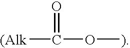
Examples of alkanoyloxy include methanoyloxy, ethanoyloxy, propanoyloxy, and the like. Examples of alkyl substituted at the terminal carbon by alkanoyloxy include 1-ethanoyloxy-1-methylethyl, propanoyloxy-1-methylethyl, and the like.

The term “alkanoyloxyaryl” includes a group of the formula 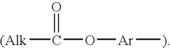
Examples of alkanoyloxyaryl include methanoyloxyphenyl, ethanoyloxyphenyl, propanoyloxyphenyl, and the like.

The term “aryl” refers to unsaturated aromatic carbocyclic radicals having a single ring, such as phenyl, or multiple condensed rings, such as naphthyl or anthryl. The term “aryl” also includes substituted aryl comprising aryl substituted on a ring by, for example, C₁₋₄ alkyl, C₁₋₄ alkoxy, amino, hydroxy, phenyl, nitro, halo, carboxyalkyl or alkanoyloxy. Aryl groups include, for example, phenyl, naphthyl, anthryl, biphenyl, methoxyphenyl, halophenyl, and the like.

The term “aryloxy” (i.e. aryl-O—) includes aryl having an oxygen atom bonded to an aromatic ring, such as, for example, phenoxy and naphthoxy.

The term “arylthio” (i.e. aryl-S—) includes aryl having a sulfur atom bonded to an aromatic ring, such as, for example, phenylthio and naphthylthio.

The term “aralkyl” refers to an aryl radical defined as above substituted with an alkyl radical as defined above (e.g. aryl-alkyl-). Aralkyl groups include, for example, phenethyl, benzyl, and naphthylmethyl.

Aryl from any of aryl, aryloxy, arylthio, aralkyl, and alkanoyloxyaryl can be unsubstituted or can be substituted on a ring by, for example, C₁₋₄ alkyl, C₁₋₄ alkoxy, amino, hydroxy, nitro, halo, or alkanoyloxy. Examples of substituted aryl include toluyl, methoxyphenyl, ethylphenyl, and the like.

The term “alkoxyalkanoyl” includes a group of the formula 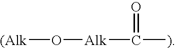
Examples of alkoxyalkanoyl include (2-acetoxy-2-methyl)propanyl, 3-ethoxy-3-propanoyl, 3-methoxy-2-propanoyl, and the like.

The term “alkoxycarbonyl” includes a group of the formula 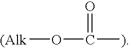
Examples of alkoxycarbonyl include methoxycarbonyl ethoxycarbonyl, propoxycarbonyl, and the like.

The term “dialkylcarbamoyloxy” includes a group of the formula 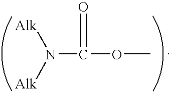
Examples of dialkylcarbamoyloxy include dimethylamino-methanoyloxy, 1-ethyl-1-methylaminomethanoyloxy, and the like. Examples of alkyl substituted at the terminal carbon by alkanoyloxy include dimethylamino-1-methylethyl, 1-ethyl-1-methylaminomethanoyloxy-1-methylethyl, and the like.

The term “halo” includes bromo, chloro, and fluoro.

In one embodiment, R₁ includes toluyl, naphthyl, phenyl, phenoxy, dimethylamino, 2,2-dimethylethyl, ethoxy, (2-acetoxy-2-methyl)propanyl, 1-ethanoyloxy-1-methylethyl, tert-butyl, acetylsalicyl, and ethanoyloxyphenyl for example.

In another embodiment R₁ groups for compounds of formula V are toluyl or naphthyl. Such R₁ groups when joined with a carbonyl group form an acyl group 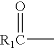
which can include toluoyl or β-naphthoyl for example. Of the toluoyl group, the p-isomer is the substituent in one embodiment.

Examples of 3-acylated analogues of pyridoxal include, but are not limited to, 2-methyl-3-toluoyloxy-4-formyl-5-hydroxymethylpyridine and 2-methyl-β-naphthoyloxy-4-formyl-5-hydroxymethylpyridine.

Examples of compounds of formula V and methods of synthesizing those compounds are described in U.S. Pat. No. 6,339,085, the disclosure of which is incorporated herein by reference.

Therapeutic compounds also include any one or more of the 3-acylated analogues of pyridoxal-4,5-aminal represented by formula VI: 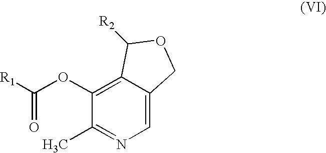
where
R₁ is alkyl or alkenyl, in which alkyl or alkenyl can be interrupted by nitrogen, oxygen, or sulfur, and can be unsubstituted or substituted at the terminal carbon with hydroxy, alkoxy, alkanoyloxy, alkanoyloxyaryl, alkoxyalkanoyl, alkoxycarbonyl, or dialkylcarbamoyloxy; R₁ is alkoxy; dialkylamino; alkanoyloxy; alkanoyloxyaryl; alkoxyalkanoyl; alkoxycarbonyl; dialkylcarbamoyloxy; R₁ is aryl, aryloxy, arylthio, or aralkyl, in which aryl can be substituted by alkyl, alkoxy, amino, hydroxy, halo, nitro, or alkanoyloxy;
R₂ is a secondary amino group;
or a pharmaceutically acceptable salt thereof.

The terms “alkyl,” “alkenyl,” “alkoxy,” “dialkylamino,” “alkanoyloxy,” “alkanoyloxyaryl,” “alkoxyalkanoyl,” “alkoxycarbonyl,” “dialkylcarbamoyloxy,” “halo,” “aryl,” “aryloxy,” “arylthio,” and “aralkyl” are as defined above for formula (V).

The term “secondary amino” group includes a group of formula VII: 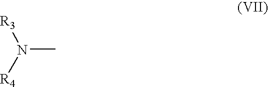
derived from a secondary amine R₃R₄NH, in which R₃ and R₄ are each independently alkyl, alkenyl, cycloalkyl, aryl, or, when R₃ and R₄ are taken together, may form a ring with the nitrogen atom and which may be interrupted by a heteroatom, such as, for example, a nitrogen, sulfur, or oxygen atom. The terms “alkyl,” “alkenyl,” and “aryl” are used as defined above in forming secondary amino groups such as, for example, dimethylamino, methylethylamino, diethylamino, dialkylamino, phenylmethylamino, diphenylamino, and the like.

The term “cycloalkyl” refers to a saturated hydrocarbon having from 3 to 8 carbon atoms, preferably 3 to 6 carbon atoms, such as, for example, cyclopropyl, cyclopentyl, cyclohexyl, and the like.

When R₃ and R₄ are taken together to form a ring with the nitrogen atom, a cyclic secondary amino group, such as, for example, piperidino, can be formed. When the cyclic secondary amino group is interrupted with a heteroatom, a group such as, for example, piperazino or morpholino can be formed.

R₁ groups for compounds of formula VI can be toluyl, naphthyl, phenyl, phenoxy, dimethylamino, 2,2-dimethylethyl, ethoxy, (2-acetoxy-2-methyl)propanyl, 1-ethanoyloxy-1-methylethyl, tert-butyl, acetylsalicyl, and ethanoyloxyphenyl for example.

In one embodiment R₁ groups can include toluyl, e.g., p-toluyl, naphthyl, tert-butyl, dimethylamino, acetylphenyl, hydroxyphenyl, or alkoxy, e.g., methoxy. Such R₁ groups when joined with a carbonyl group form an acyl group 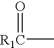
which can include toluoyl, β-naphthoyl, pivaloyl, dimethylcarbamoyl, acetylsalicyloyl, salicyloyl, or alkoxycarbonyl. In one embodiment, R₂, the preferred secondary amino group can be morpholino.

Examples of 3-acylated analogues of pyridoxal-4,5-aminal include, but are not limited to, 1-morpholino-1,3-dihydro-7-(p-toluoyloxy)-6-methylfuro(3,4-c)pyridine; 1-morpholino-1,3-dihydro-7-(β-naphthoyloxy)-6-methylfuro(3,4-c)pyridine; 1-morpholino-1,3-dihydro-7-pivaloyloxy-6-methylfuro(3,4-c)pyridine; 1-morpholino-1,3-dihydro-7-carbamoyloxy-6-methylfuro(3,4-c)pyridine; and 1-morpholino-1,3-dihydro-7-acetylsalicyloxy-6-methylfuro(3,4-c)pyridine.

Examples of compounds of formula VI and methods of synthesizing those compounds are described in U.S. Pat. No. 6,339,085, the disclosure of which is incorporated herein by reference.

Therapeutic compounds include any one or more pyridoxal phosphonate analogues represented by the formula VII: 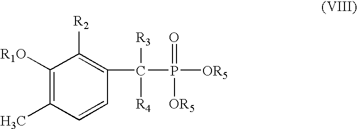
where
R₁ is hydrogen or alkyl;
R₂ is —CHO; —CH₂OH; —CH₃; —CO₂R₆ in which R₆ is hydrogen, alkyl, or aryl; and —CH₂₁₃O-alkyl- in which alkyl is covalently bonded to the oxygen at the 3-position instead of R₁;
R₃ is hydrogen; and R₄ is hydroxy, halo, alkoxy, alkanoyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable salt thereof.

The terms “alkyl,” “alkoxy,” “alkanoyloxy,” “halo,” “aryl,” and “aralkyl” are as defined above for formula V.

The term “alkylamino” refers to —NH-alkyl with alkyl as defined above. Alkylamino groups include those with 1-6 carbons in a straight or branched chain, such as, for example, methylamino, ethylamino, propylamino, and the like.

The term “arylamino” refers to —N-aryl with aryl as defined above. Arylamino includes —NH-phenyl, —NH-biphenyl, —NH-4-methoxyphenyl, and the like.

Examples of compounds of formula VIII include those where R₁ is hydrogen, or those where R₂ is —CH₂OH, or —CH_{2—}O-alkyl- in which alkyl is covalently bonded to the oxygen at the 3-position instead of R₁, or those where R₃ is hydrogen and R₄ is F, MeO— or CH₃C(O)O—, or those where R₅ is alkyl or aralkyl. Additional examples of compounds of formula VIII include those where R₃ and R₄ are F, or those where R₅ is t-butyl or benzyl.

Therapeutic compounds further include any one or more pyridoxal phosphonate analogues represented by the formula IX: 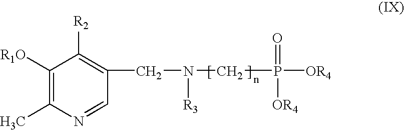
in which
R₁ is hydrogen or alkyl;
R₂ is —CHO, —CH₂OH, —CH₃ or —CO₂R₅ in which R₅ is hydrogen, alkyl, or aryl; —CH_{2—}O-alkyl- in which alkyl is covalently bonded to the oxygen at the 3-position instead of R₁;
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl;
n is 1 to 6;
   or a pharmaceutically acceptable salt thereof.

The terms “alkyl,” “aryl,” and “aralkyl” are as defined above for formula V.

Examples of compounds of formula IX include those where R₁ is hydrogen, or those where R₂ is —CH₂OH, or —CH_{2—}O-alkyl- in which alkyl is covalently bonded to the oxygen at the 3-position instead of R₁, or those where R₃ is hydrogen, or those where R₄ is alkyl or hydrogen. Additional examples of compounds of formula IX include those where R₄ is ethyl.

Therapeutic compounds further include any one or more pyridoxal phosphonate analogues represented by the formula X: 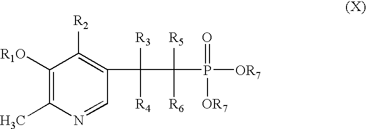
in which
R₁ is hydrogen or alkyl;
R₂ is —CHO, —CH₂OH, —CH₃ or —CO₂R₈ in which R₈ is hydrogen, alkyl, or aryl; or
R₂ is —CH_{2—}O-alkyl- in which alkyl is covalently bonded to the oxygen at the 3-position instead of R₁;
R₃ is hydrogen and R₄ is hydroxy, halo, alkoxy or alkanoyloxy; or
R₃ and R₄ can be taken together to form ═O;
R₅ and R₆ are hydrogen; or
R₅ and R₆ are halo;
R₇ is hydrogen, alkyl, aryl, aralkyl, or —CO₂R₈ in which R₈ is hydrogen, alkyl, aryl, or aralkyl;
or a pharmaceutically acceptable salt thereof.

The terms “alkyl,” “alkoxy,” “alkanoyloxy,” “halo,” “aryl,” and “aralkyl” are as defined above for formula V.

Examples of compounds of formula IX include those where R₁ is hydrogen, or those where R₂ is —CH₂OH, or —CH_{2—}O-alkyl- in which alkyl is covalently bonded to the oxygen at the 3-position instead of R₁, or those where R₃ and R₄ taken together form ═O, or those where R₅ and R₆ are F, or those where R₇ is alkyl. Additional examples of compounds of formula IX include those where R₄ is OH or CH₃C(O)O—, those where R₇ is ethyl.

Pharmaceutically acceptable salts of the compounds of formulas I, II, III, IV, V, VI, VII, VIII, IX, or X include acid addition salts derived from nontoxic inorganic acids such as hydrochloric, nitric, phosphoric, sulfuric, hydrobromic, hydriodic, hydrofluoric, phosphorus, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977)).

The salts of the basic compounds are prepared by contacting the free base form with a sufficient amount of a desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically accepted salts of the compounds of formulas VIII, IX, and X include metals such as alkali and alkaline earth metals. Examples of metals used as cations are sodium, potassium, magnesium, calcium, and the like. Also included are heavy metal salts such as for example silver, zinc, cobalt, and cerium.

Syntheses

To prepare a compound of formula VIII, 3,4-isopropylidenepyridoxine-5-al can be treated with a phosphonating agent, such as, a metal salt of di-tert-butyl phosphite or dibenzyl phosphite or diphenyl phosphite, to give protected alpha-hydroxyphosphonates. The protected alpha-hydroxyphosphonates can be treated with an acylating agent in an aprotic solvent, such as acetic anhydride in pyridine, or with an alkylating agent, such as methyl iodide and sodium hydride in tetrahydrofuran (THF), to give alpha-alkylcarbonyloxy or alpha-alkyloxyphosphonates esters respectively.

Alternatively the protected alpha-hydroxyphosphonates can be treated with an agent to convert the hydroxyl group to a halogen, such as conversion to a fluoro group with DAST (diethylaminosulfurtrifluoride), to prepare the alpha-halophosphonate esters. The isopropylidene protecting group is removed from the fully protected alpha-substituted phosphonates by reacting them with water and an acid, such as 20% water in acetic acid, to prepare the pyridoxine-alpha-substituted phosphonate esters. The ester groups can be removed from the phosphonate groups of the pyridoxine-alpha-substituted phosphonate esters by further treating them with acid in water, such as 20% water in acetic acid, to give the corresponding phosphonic acids as can be seen in the following scheme.

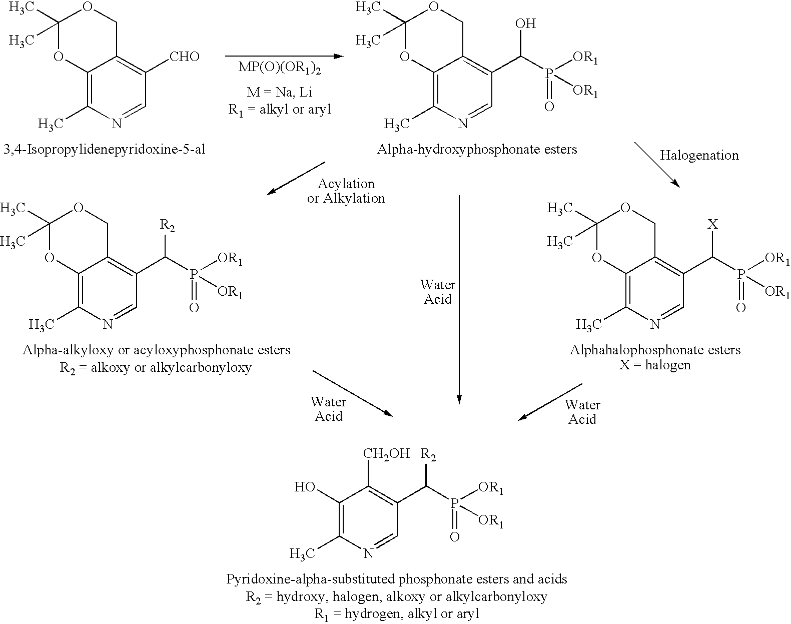

Alternatively, to prepare a compound of formula VIII, 3,4-isopropylidenepyridoxine-5-halide can be treated with a phosphonating agent, such as, a metal salt of di-tert-butyl phosphite or dibenzyl phosphite or diphenyl phosphite, to give protected phosphonates. The protected phosphonates are treated with a base, such as sodium hexamethyldisilazane (NaHMDS), and a halogenating agent, such as N-fluorobenzenesulfonimide (NFSi), to provide the dihalophosphonates as can be seen in the following scheme.

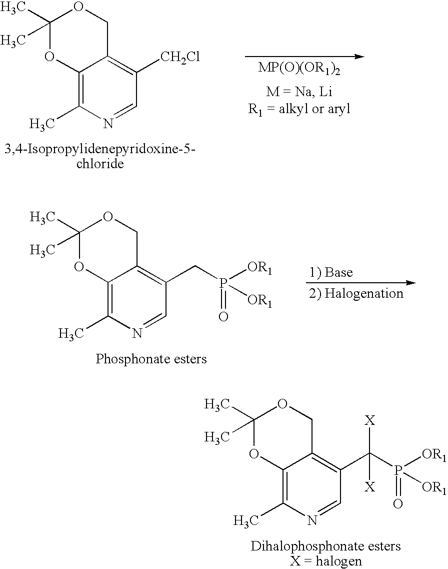

Alternatively, to prepare a compound of formula VIII, 3,4-isopropylidenepyridoxine-5-al can be treated with an amine, such as p-methoxyaniline or p-aminobiphenyl, and a phosphonating agent, such as, a metal salt of di-tert-butyl phosphite, dibenzyl phosphite or diphenyl phosphite, to give protected aminophosphonates as can be seen in the following scheme.

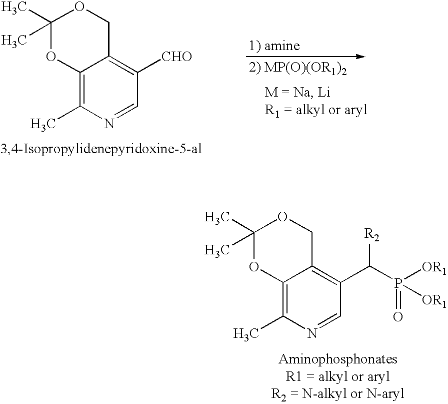

To prepare a compound of formula IX, 3,4-isopropylidenepyridoxine-5-amine can be used as a starting material. The amine is treated with a haloalkylphosphonate diester, such as diethyl bromomethylphosphonate, to give 5′-phosphonoazaalkylpyridine diesters. Reaction of the 3,4-isopropylidene-5′-phosphonoazaalkylpyridoxine diesters with a trialkylsilyl halide, such as trimethylsilyl bromide, in an aprotic solvent, such as acetonitrile, removes the ester groups of the phosphonate diester to provide the corresponding free 3,4-isopropylidene-5′-phosphonoazaalkylpyridoxine diacid. The acetonide protecting group on the 3 and 4 position of the pyridoxine ring on the 3,4-isopropylidene-5′-phosphonoazaalkylpyridoxine diacid can be removed by reaction with acid and water, such as 20% water in acetic acid as can be seen in the following scheme.

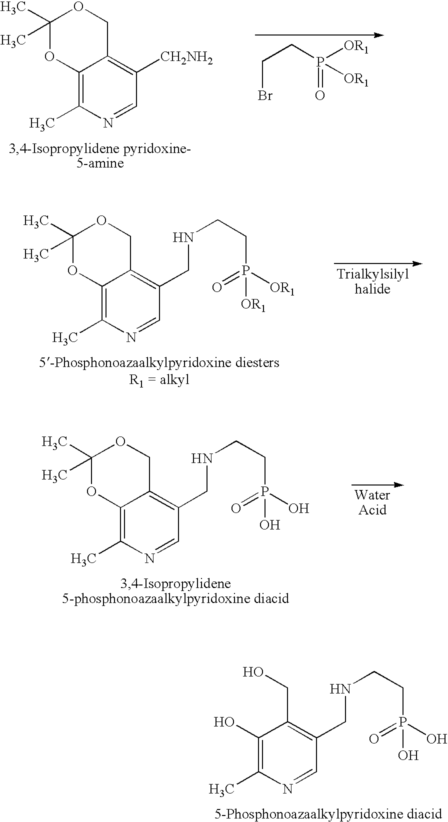

To prepare a compound of formula X, 3,4-isopropylidenepyridoxine-5-al can be reacted with a metal salt of a methyl, or dihalomethyl, phosphonate diester to produce 5′-phosphonoalkylpyridoxine diesters. The 5′-hydroxyl group of this product is acylated by an acylating agent, such as acetic anhydride in pyridine, to provide the corresponding O-acyl derivatives respectively, or oxidized to the keto functional group by an oxidizing agent, such as manganese dioxide. The blocking group at the 3 and 4 positions and the phosphonate ester groups of the hydroxy, alkylcarbonyloxy and keto phosphonate diesters are hydrolysed by reaction with acid and water, such as 20% water in acetic acid, to provide the corresponding phosphonate diesters, without the blocking group at the 3 and 4 position. These reactions are illustrated in the following scheme. 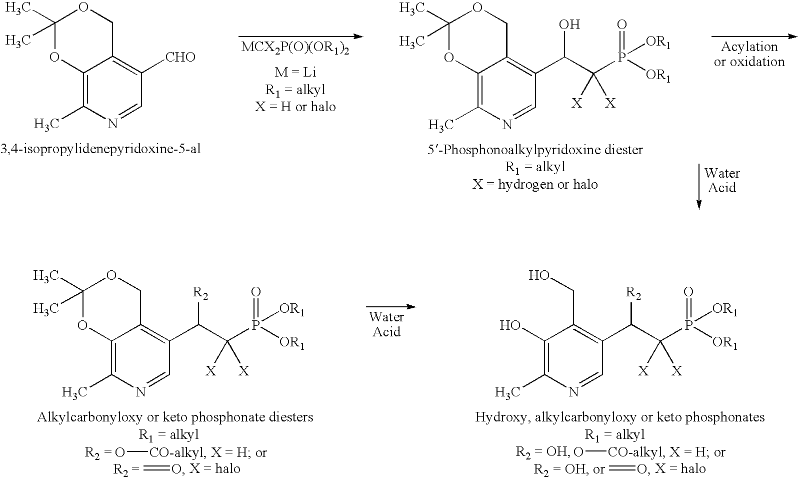
Pharmaceutical Composition Suitable for Use with Methods of the Invention

A therapeutic compound as defined above can be formulated into a pharmaceutical composition for use in methods of the invention. A pharmaceutical composition is suitable for modulation of cell death.

A pharmaceutical composition comprises a pharmaceutically acceptable carrier and at least one therapeutic compound of formula I, II, III, IV, V, VI, VIII, IX, or X or a pharmaceutically acceptable salt thereof. A pharmaceutically acceptable carrier includes, but is not limited to, physiological saline, ringers, phosphate-buffered saline, and other carriers known in the art. Pharmaceutical compositions can also include additives, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives can be chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier and at least one therapeutic compound of formula I, II, III, IV, V, VI, VIII, IX, or X or a pharmaceutically acceptable salt thereof are known to those of skill in the art.

All methods can include the step of bringing the compound of the invention in association with the carrier and additives. The formulations generally are prepared by uniformly and intimately bringing the compound of the invention into association with a liquid carrier or a finely divided solid carrier or both, and then, if necessary, shaping the product into the desired unit dosage form.

Generally, a solution of a therapeutic compound, for example PLP, may be prepared by simply mixing PLP with a pharmaceutically acceptable solution, for example, buffered aqueous saline solution at a neutral or alkaline pH (because PLP is essentially insoluble in water, alcohol, and ether), at a temperature of at least room temperature and under sterile conditions. In one embodiment, the PLP solution is prepared immediately prior to administration to the mammal. However, if the PLP solution is prepared at a time more than immediately prior to the administration to the mammal, the prepared solution can be stored under sterile, refrigerated conditions. Furthermore, because PLP is light sensitive, the PLP solution can be stored in containers suitable for protecting the PLP solution from the light, such as amber-colored vials or bottles.

A pharmaceutical composition or therapeutic compound can be administered enterally or parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition or therapeutic compound should be at or near body temperature.

Methods of Treatment

A physician or veterinarian of ordinary skill can readily determine a subject who is or may be suffering from a disease state or traumatic injury that could implicate cell death. Regardless of the route of administration selected, the therapeutic compounds of formula I, II, III, IV, V, VI, VIII, IX, or X or a pharmaceutically acceptable salt thereof can be formulated into pharmaceutically acceptable unit dosage forms by conventional methods known to the pharmaceutical art. An effective but nontoxic quantity of the compound can be employed in treatment.

The therapeutic compound of formula I, II, III, IV, V, VI, VIII, IX, or X or a pharmaceutically acceptable salt thereof can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and other administrative methods known in the art.

Although it is possible for a therapeutic compound of formula I, II, III, IV, V, VI, VIII, IX, or X or a pharmaceutically acceptable salt thereof as described above to be administered alone in a unit dosage form, preferably the compound is administered in admixture as a pharmaceutical composition.

The ordinarily skilled physician or veterinarian will readily determine and prescribe a therapeutically effective amount of the at least one therapeutic compound of formula I, II, III, IV, V, VI, VIII, IX, or X or a pharmaceutically acceptable salt thereof to modulate cell death. In so proceeding, the physician or veterinarian could employ relatively low dosages at first, subsequently increasing the dose until a maximum response is obtained. Typically, the particular disease, the severity of the disease, the extent of cell death or stress, the compound to be administered, the route of administration, and the characteristics of the mammal to be treated, for example, age, sex, and weight, can be considered in determining the effective amount to administer. In one embodiment of the invention, a therapeutic amount is in a range of about 0.1-100 mg/kg of a patient's body weight, in another embodiment in the range of about 0.5-50 mg/kg of a patient's body weight, per daily dose. The compound can be administered for periods of short or long duration. Although some individual situations can warrant to the contrary, short-term administration, for example, 30 days or less, of doses larger than 25 mg/kg of a patient's body weight is chosen when compared to long-term administration. When long-term administration, for example, months or years, is utilized, the suggested dose generally should not exceed 25 mg/kg of a patient's body weight.

A therapeutically effective amount of a therapeutic compound of formula I, II, III, IV, V, VI, VII, VIII, IX, or X or a pharmaceutically acceptable salt thereof for modulating cell death that may be caused by the above-identified diseases or symptoms thereof can be administered prior to, concurrently with, or after the onset of the disease or symptom.

A therapeutic compound of the invention can be administered concurrently with or subsequent to compounds that are already known to be suitable for treating the disease state or traumatic injury that may be causing the cell death. “Concurrent administration” and “concurrently administering” as used herein includes administering a therapeutic compound and a known therapy in admixture such as, for example, in a pharmaceutical composition or in solution, or as separate components, such as, for example, separate pharmaceutical compositions or solutions administered consecutively, simultaneously, or at different times but not so distant in time such that the therapeutic compound and the known therapy cannot interact and a lower dosage amount of the active ingredient cannot be administered.

This invention will be further characterized by the following examples. These examples are not meant to limit the scope of the invention, which has been fully set forth in the foregoing description. Variations within the scope of the invention will be apparent to those skilled in the art.

### EXAMPLES

All reagents used in the following Examples can be purchased from Aldrich Chemical Company (Milwaukee, Wis. or Allentown, Pa.).

### Example 1

### Synthesis of di-t-butyl (α⁴,3-O-isopropylidene-3-hydroxy-4-hydroxymethyl-2-methyl-5-pyridyl)hydroxymethylphosphonate

Di-tert-butyl phosphite (16.3 g, 84 mmol) was added to a solution of NaH (3.49 g, 60%, 87.2 mmol) in TBF (60 mL) under nitrogen at 0° C. The temperature of the resulting solution was raised to room temperature and the solution stirred for 15 min, then cooled to 0° C. again. To this solution, (α⁴,3-O-isopropylidene-3-hydroxy-4-hydroxymethyl-2-methyl-5-pyridyl)methanal (Kortynk et al., J. Org. Chem., 29, 574-579 (1964)) (11.41 g, 55.05 mmol) in THF (30 mL) was slowly added then the temperature raised to room temperature again and stirring continued for 2 h The reaction was quenched by adding saturated NaHCO₃ (40 ml), and diluted with diethyl ether (200 mL). The ether layer was separated, washed with saturated aqueous NaHCO₃ (40 ml, 5%), then saturated brine (3×20 mL). The ether layer was dried (MgSO₄), filtered and evaporated to give crude product as a colorless solid. This solid was washed with hexane to remove the oil (from the NaH) and unreacted phosphite. The solid was recrystallized from a mixture of diethyl ether:hexane:ethyl acetate (230 mL:70 mL:15 mL). The colorless crystal (17.9 g, 81%) were filtered and washed with hexane.

¹H NMR (CDCl₃): 1.42 (9H, d), 1.46 (9H, d), 1.51 (6H, d), 2.38 (3H, s), 4.70 (1H, d), 4.89-5.13 (2H, m), 8.11 (1H, s).

³¹P NMR (H-decoupled, CDCl₃): 13.43 (s).

This structure can be represented by formula:

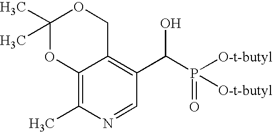

### Example 2

### Synthesis of dibenzyl (α⁴,3-O-isopropylidene-3-hydroxy-4-hydroxymethyl-2-methyl-5-pyridyl)hydroxymethylphosphonate

Dibenzyl phosphite (1.89 g, 9.62 mmol) was mixed with the (α⁴,3-O-isopropylidene-3-hydroxy-4-hydroxymethyl-2-methyl-5-pyridyl)methanal (Kortynk et al., J. Org. Chem., 29, 574-579 (1964)) (1.00 g, 4.81 mmol) and stirred at room temperature for an hour. To this thick syrup was added activated basic alumina (1 g). The reaction mixture was then stirred at 80° C. for one hour. The reaction mixture was diluted with dichloromethane (50 mL), and filtered through Celite to remove alumina. The dichloromethane solution was washed with saturated, aqueous NaHCO₃ (20 mL), then saturated brine (3×10 mL). The dichloromethane layer was dried (MgSO₄), filtered and evaporated to give crude product as a colorless solid. The crude product was purified by silica gel column chromatography, using ether: hexanes (1:2) as eluent to give 1.3 g (58%).

¹H NMR (CDCl₃): 1.30 (3H, s), 1.45 (3H, s), 2.30 (3H, s), 4.86-4.99 (7H,s), 7.18-8.07 (10H, s), 8.08 (1H, s).

This structure can be represented by formula:

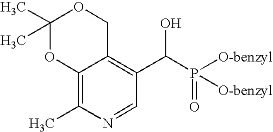

### Example 3

### Synthesis of (3-hydroxy-4-hydroxymethyl-2-methyl-5-pyridyl)hydroxymethyl phosphonic Acid

The product of Example 1 above, of formula V, (10 g, 24.9 mmol) was dissolved in acetic acid (80% in water, 100 ml) and heated at 60° C. for 1 d. Colorless precipitate was formed, however, the reaction was not complete. Another 50 ml of 80% acetic acid in water was added to the mixture and the mixture stirred at 60° C. for another day. The solid was filtered off, washed with cold water, then methanol and dried to give a colorless solid (4.78 g, 77%).

¹H NMR (D₂O): 2.47 (3H, s), 4.75-4.79 (2H, m), 5.15-5.19 (1H, d), 7.82 (1H, s).

³¹P NMR (H-decoupled D₂O): 14.87 (s).

This structure can be represented by formula:

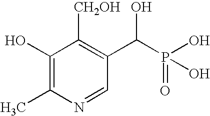

### Example 4

### Synthesis of dibenzyl (α⁴,3-O-isopropylidene-3-hydroxy-4-hydroxymethyl-2-methyl-5-pyridyl)fluoromethylphosphonate

The protected alpha-hydroxy phosphonate from Example 2 above of structure VI (1.0 g, 2.49 mmol) was dissolved in dichloromethane (10 mL), and the solution cooled to −78° C. To this solution was added diethylaminosulfurtrifluoride (DAST) (0.8 g, 4.98 mmol). The reaction was stirred at −78° C. under nitrogen for 20 minutes then allowed to stand at room temperature overnight. The reaction mixture was diluted with dichloromethane (50 ml), and washed with saturated, aqueous NaHCO₃ (125 mL). The dichloromethane layer was dried (MgSO₄), filtered and evaporated to give crude fluorophosphonate as a yellow solid. The crude product was purified by silica gel column chromatography, using ethyl acetate:hexanes (2:1) as the eluent to give 600 mg (60%).

¹H NMR (CDCl₃): 1.42 (3H, s), 1.52 (3H, s), 2.40 (3H, s), 4.91-4.97 (6H, m), 5.64-5.61 (1H, dd), 7.23-7.34 (10H, m), 8.01 (1H, s).

³¹P NMR (H-decoupled, F-coupled, CDCl₃): 16.36-16.08 (d).

This structure can be represented by formula:

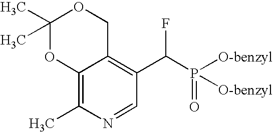

### Example 5

### Synthesis of di-t-butyl (α⁴,3-O-isopropylidene-3-hydroxy-4-hydroxymethyl-2-methyl-5-pyridyl)fluoromethylphosphonate

The protected alpha-hydroxy phosphonate from Example 1 of structure V (3 g, 7.55 mmol) was dissolved in dichloromethane (30 mL), and the solution cooled to −78° C. To this solution was added diethylaminosulfurtrifluoride (DAST) (1.22 g, 7.57 mmol). The reaction was stirred at −78° C. under nitrogen for 5 minutes, quenched by addition of saturated, aqueous NaHCO₃ (2 mL) then allowed to warm room temperature. The reaction mixture was diluted with dichloromethane (50 ml), and washed with saturated, aqueous NaHCO₃ (2×20 mL). The dichloromethane layer was dried (MgSO₄), filtered and evaporated to give crude fluorophosphonate. The crude product was purified by silica gel column chromatography, using ethyl acetate:hexanes (1:1) as the eluent to give 350 mg (12%).

¹H NMR (CDCl₃): 1.44 (9H, s), 1.46 (9H, s), 1.52 (3H, s), 1.56 (3H, s), 2.41 (3H, s), 4.98-5.14 (2H, m), 5.32-5.52 (1H, dd), 8.03 (1H, s).

³¹P NMR (H-decoupled, F-coupled, CDCl₃): 6.53, 7.24.

¹⁹F NMR (H-decoupled, CDCl₃): −202.6, −203.0

This structure can be represented by formula:

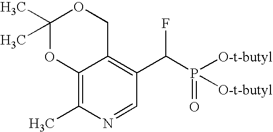

### Example 6

### Synthesis of di-t-butyl (3-hydroxy-4-hydroxymethyl-2-methyl-5-pyridyl)fluoromethyl phosphonate

The protected di-t-butyl alpha-fluoro phosphonate from Example 5 of structure IX (3.2 g 7.8 mmol) was dissolved in acetic acid (80% in water, 50 ml) and heated at 60° C. for 24 hours. The pale yellow solid was filtered off, washed with cold water and methanol, and then dried to give a creamy solid (2.21 g, 70%).

¹H NMR (CDCl₃): 1.41 (9H, s), 1.44 (9H, s), 1.49 (3H, s), 1.51 (3H, s), 2.42 (3H, s), 4.99-5.07 (2H, m), 5.33-5.51 (1H, d,d), 8.04 (1H, s).

³¹P NMR (H-decoupled, F-Coupled, CDCl₃): 7.10-7.80 (d).

¹⁹F NMR (H, P-Coupled, CDCl₃): −203.07 to −202.61 (dd).

This structure can be represented by formula:

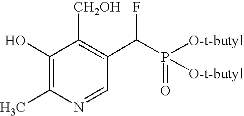

### Example 7

### Synthesis of (3-hydroxy-4-hydroxymethyl-2-methyl-5-pyridyl)fluoromethyl phosphonic acid

The protected di-t-butyl alpha-fluoro phosphonate from Example 5 of structure IX (200 mg, 0.5 mmol) was dissolved in acetic acid (80% in water, 15 ml) and heated at 75° C. for 24 hours. The solvent was removed by evaporation on a rotary evaporator using toluene to codistill the water. The crude product (183 mg) was purified by column chromatography on silica using chloroform:methanol:water (65:35:2) as eluent to give 60 mg (55%).

¹H NMR (D₂O): 2.46 (3H, bs), 4.65-4.90 (2H, dd), 5.81-6.01 (1H, dd), 7.74 (1H, bs).

³¹P NMR (H-decoupled, F-Coupled, CDCl₃): 9.3 (d).

¹⁹F NMR (H, P-Coupled, CDCl₃): −197 to −196 (dd).

This structure can be represented by formula:

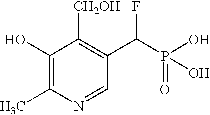

### Example 8

### Synthesis of di-t-butyl (α⁴,3-O-isopropylidene-3-hydroxy-4-hydroxymethyl-2-methyl-5-pyridyl)acetoxymethylphosphonate

The product of Example 1 above, of formula V (1.0 g, 2.49 mmol) was dissolved in dichloromethane (20 mL), the solution cooled to −5° C., and pyridine (2 mL) added, followed by acetic anhydride (1 mL). The reaction temperature was slowly allowed to reach room temperature. After one hour, the reaction was quenched by adding dilute aqueous hydrochloric acid (10%, 75 mL), and then diluted with dichloromethane (25 mL). After separation of the aqueous layer the methylene chloride layer washed with saturated NaHCO₃ (2×20 mL). The dichloromethane layer was dried (MgSO₄), filtered and evaporated to give crude alpha acetoxy phosphonate as a colorless solid. The crude product was purified by silica gel column chromatography, using ethyl acetate:hexanes (2:1) as the eluent to give the product in good yield.

¹H NMR (CDCl₃): 1.31 (9H, d), 1.36 (9H, d), 1.49 (6H, d), 2.1 (3H s), 2.38 (3H, s), 5.04 (2H, d), 5.72-5.76 (1H, d), 8.11 (1H, s).

³¹P NMR (H-decoupled, CDCl₃): 13.43 (s).

This structure can be represented by formula:

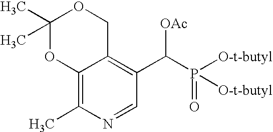

### Example 9

### Synthesis of di-t-butyl (α⁴,3-O-isopropylidene-3-hydroxy-4-hydroxymethyl-2-methyl-5-pyridyl)methoxymethylphosphonate

The product of Example 1 above, of formula V (300 mg, 0.75 mmol) was dissolved in 15 ml of THF and reaction vessel was purged with N₂ gas. Sodium hydride (21 mg, 0.9 mmol) was added, and the solution stirred for 5 minutes before cooling to 0° C. Methyl iodide (160 mg, 1.1 mmol) was then injected and reaction vessel was gradually allowed to reach room temperature. TLC (ethyl acetate) indicated that the reaction was complete in 3 hours. The solution was diluted with methylene chloride (250 mL), washed with dilute, aqueous HCL (10%, 100 mL), then saturated, aqueous NaHCO₃, dried MgSO₄) and evaporated. The crude product was chromatographed on silica gel using ethyl acetate/hexanes (1:1) as the eluent to give 132 mg (32%).

¹H NMR (CDCl₃): 1.41 (18H, s), 1.51 (3H, s), 1.54 (3H, s), 2.40 (3H, s), 3.33 (3H, s), 4.20-4.26 (1H, d), 5.05 (2H, bs), 8.01 (1H, s).

³¹P NMR (H-decoupled, CDCl₃): 10.88 (s).

This structure can be represented by formula:

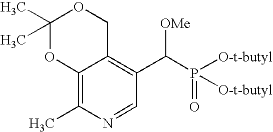

### Example 10

### Synthesis of (3-hydroxy-4-hydroxymethyl-2-methyl-5-pyridyl)acetoxymethyl phosphonic Acid

The product of Example 8 above, of formula XII, (50 mg, 0.11 mmol) was added to acetic acid (80% in water) and stirred for 24 hours at 60° C. The solvent was removed by evaporation on a rotary evaporator using toluene to codistill the water. The crude product was purified by chromatography on silica gel column using CH₂Cl₂/MeOH/H₂O (65:35:4) as eluent to give 22.8 mg (76%).

¹H NMR (D₂O): 2.23 (3H, s), 2.51 (3H, s), 4.6-5.1 (2H, m), 6.1 (1H, d), 7.85 (1H, s).

This structure can be represented by formula:

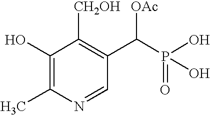

### Example 11

### Synthesis of (3-hydroxy-4-hydroxymethyl-2-methyl-5-pyridyl)methoxymethyl phosphonic Acid

The product of Example 9 above, of formula XIII (132 mg, 0.32 mmol) was dissolved in acetic acid (80% in water, 25 mL) and stirred at 60° C. for 24 hours. The solvent was removed by evaporation on a rotary evaporator using toluene to codistill the water. The crude product was purified by chromatography on silica gel column using CH₂Cl₂/MeOH/H₂O (65:35:4) as eluent to give the product in good yield.

¹H NMR (D₂O): 2.52 (3H, s), 3.32 (3H, s), 4.47-4.88 (2H, m), 7.87 (1H, s).

³¹P NMR (H-decoupled, D₂O): 13.31 (s)

This structure can be represented by formula:

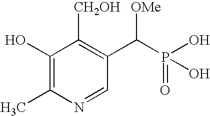

### Example 12

### Synthesis of di-t-butyl (α⁴,3-O-isopropylidene-3-hydroxy-4-hydroxymethyl-2-methyl-5-pyridyl)difluoromethylphosphonate

To a solution of dibenzyl (α⁴,3-O-isopropylidene-3-hydroxy-4-hydroxymethyl-2-methyl-5-pyridyl)methylphosphonate (115 mg, 0.253 mmol) in THF (10 mL) was added NaHMDS (1 M, 0.56 mL, 0.56 mmol). The reaction mixture was cooled to −78° C. After 15 minutes, NFSi (237 mg, 0.75 mmol) was added to the reaction mixture. The temperature of the reaction mixture was slowly warmed to −20° C. The solution was diluted with Et₂O, washed with saturated NaHCO₃, water and brine, dried (MgSO₄) and evaporated. The crude product was chromatographed on silica using ethyl acetate:hexanes (2:1) as eluent to give the dibenzyl (α⁴,3-O-isopropylidene-3-hydroxy-4-hydroxymethyl-2-methyl-5-pyridyl)difluoromethylphosphonate in good yields.

¹H NMR (CDCl₃) 1.53 (s, 6H), 2.45 (d, 3H), 5.34 (d, 2H), 7.09-7.39 (m, 14H), 8.29 (s,1H).

³¹P NMR (CDCl₃) -2.15 (t).

¹⁹F NMR (CDCl₃) -105.7 (d).

This structure can be represented by formula:

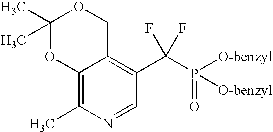

### Example 13

### Synthesis of di-t-butyl (α⁴,3-O-isopropylidene-3-hydroxy-4-hydroxymethyl-2-methyl-5-pyridyl)(4-biphenylamino)methylphosphonate

The (α⁴,3-O-isopropylidene-3-hydroxy-4-hydroxymethyl-2-methyl-5-pyridyl)methanal (Kortynk et al., J. Org. Chem., 29, 574-579 (1964)) (424 mg, 2.19 mmol) and 4-aminobiphenyl (360 mg, 2.12 mmol) was refluxed in benzene (20 mL) under nitrogen, using a Dean-Stark trap to remove water, for 15 hours. The crude reaction mixture was evaporated, dissolved in THF (20 mL) and added to a flask containing di-t-butyl phosphite (955 mg, 5.12 mmol) in THF (20 mL) and NaH (270 mg, 57% in oil, 6.41 mmol) and stirred at 0° C. for two hours. The solution was diluted with Et₂O, washed with saturated, aqueous NaHCO₃ (40 mL), brine (20 mL), dried (MgSO₄) and evaporated. The crude product was chromatographed on silica gel using hexane:diethyl ether (2:1) to give di-t-butyl (α⁴,3-O-isopropylidene-3-hydroxy-4-hydroxymethyl-2-methyl-5-pyridyl)(4-biphenylamino)methylphosphonate in modest yields.

¹H NMR (CDCl₃) 8.40 (1H, d,), 7.50-7.41 (2H, m), 7.40-7.30 (4H, m), 7.28-7.10 (1H, m), 6.54 (1H, d), 5.24 (1H, dd,), 5.07 (1H, dd,), 4.65 (1H, dd,), 4.44 (1H, dd,), 2.40 (3H, d), 1.58 (3H, s), 1.49 (3H, s), 1.43 (9H, s), 1.41 (9H, s).

³¹P NMR (H-decoupled, CDCl₃): 13.1 (s).

This structure can be represented by formula:

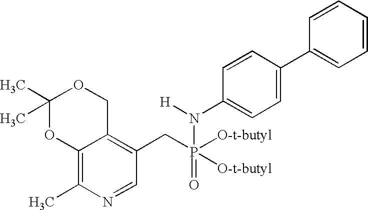

### Example 14

### Synthesis of di-t-butyl (α⁴,3-O-isopropylidene-3-hydroxy-4-hydroxymethyl-2-methyl-5-pyridyl)(4-methoxyphenylamino)methylphosphonate

(α⁴,3-O-Isopropylidene-3-hydroxy-4-hydroxymethyl-2-methyl-5-pyridyl)methanal (Kortynk et al., J. Org. Chem., 29, 574-579 (1964)) (2.5 g, 12.1 mmol) and 4-aminoanisole (1.41 g, 11.4 mmol) was refluxed in benzene (100 mL) under nitrogen, using a Dean-Stark trap to remove water, for 15 hours. The reaction mixture was evaporated to give 3.02 g of crude imine. The crude imine (370 mg, 1.19 mmol) was dissolved in THF (20 mL) and added to a flask containing di-t-butyl phosphite (955 mg, 5.1 mmol) in THF (20 mL) and NaH (208 mg, 57% in oil, 4.94 mmol) and stirred at 0° C. for two hours and at room temperature for 24 hours. The solution was diluted with Et₂O, washed with saturated, aqueous NaHCO₃ (40 mL), brine (40 mL), dried (MgSO₄) and evaporated. The crude product was chromatographed on silica gel using hexane:diethyl ether (2:1) to give di-t-butyl (′⁴,3-O-isopropylidene-3-hydroxy-4-hydroxymethyl-2-methyl-5-pyridyl)(4-methoxyphenylamino)methylphosphonate in modest yields.

¹H NMR (CDCl₃) 8.09 (1H, d), 6.70-6.60 (2H, m), 6.47-6.36 (2H, m), 5.18 (1H, dd), 4.98 (1H, dd), 4.36-4.20 (2H, m), 3.65 (3H, s), 2.35 (3H, s), 1.54 (3H, s), 1.45 (3H, s), 1.39 (9H, s), 1.38 (9H, s).

³¹P NMR (decoupled, CDCl₃): δ 13.5 ppm.

This structure can be represented by formula:

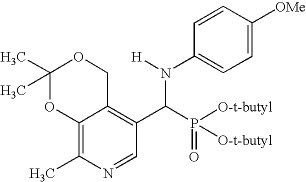

### Example 15

### Synthesis of di-t-butyl (α⁴,3-O-isopropylidene-3-hydroxy-4-hydroxymethyl-2-methyl-5-pyridyl)-3-azabutylphosphonate

(α⁴,3-O-Isopropylidene-3-hydroxy-4-hydroxymethyl-2-methyl-5-pyridyl)methylbromide (Imperalli et al, J. Org. Chem., 60, 1891-1894 (1995)) (1.08 g. 4.0 mmol) in anhydrous DMF (20 ml) was treated with sodium azide (260 mg, 4.0 mmol) at room temperature. After one hour stirring at room temperature, the solution was extracted with diethyl ether (5×20 mL). The combined extracts were washed with water (10 mL), and brine (10 mL) and dried (MgSO₄). The solvent was evaporated and the crude product was purified by chromatography on silica gel using ethyl ether:hexanes (2:1) as eluent to give the azide as a colorless liquid (552 mg, 60%).

¹H NMR (CDCl3, TMS) 1.57 (s, 6H), 2.42 (s, 3H), 4.23 (s, 2H), 4.86 (s, 2H), 7.96 (s, 1H).

The purified azide (100 mg, 0.4 mmol) was dissolved in 95% ethanol and hydrogenated at 1 atm in presence of Lindlar catalyst (50 mg) for one hour. The catalyst was removed by filtration (Celite), and the solvent removed to give the crude amine. Purification by chromatography on silica gel using CH₂Cl₂:MeOH (5:1) as eluent gave the product (80 mg, 82%) 1HNMR (CD₂Cl₂) 1.53 (s, 6H), 2.34 (s, 3H), 3.72 (s, 2H), 4.91 (s, 2H), 5.31 (s, 2H), 7.93 (s, 1H).

The (α⁴,3-O-Isopropylidene-3-hydroxy-4-hydroxymethyl-2-methyl-5-pyridyl)methylamine, from above, (416 mg, 2 mmol) was heated in saturated, aqueous sodium bicarbonate solution (10 mL) to 95° C., followed by slow addition of diethyl 2-bromoethylphosphonate (0.09 mL, 0.5 mmol) and the reaction stirred at 95° C. overnight. The solution is evaporated using toluene to codistill the water. The crude product is triturated with ethyl acetate to dissolve the crude organic product. Chromatography on silica gel using methylene chloride:methanol:hexanes (5:1:5) gave 76 mg (41%).

¹Hnmr (CDCl₃, TMS) 1.27 (t, 6H), 1.51 (s, 6H), 1.91 (t, 2H), 2.35 (s, 3H), 2.85 (t, 2H), 3.62 (s, 2H), 4.03 (m, 4H), 4.91 (s, 2H), 7.88 (s, 1H).

³¹P NMR (H-decoupled, CDCl₃): 31.00 (s).

This structure can be represented by formula:

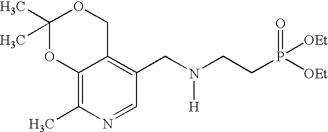

### Example 16

### Synthesis of (α⁴,3-O-isopropylidene-3-hydroxy-4-hydroxymethyl-2-methyl-5-pyridyl)-3-azabutylphosphonic acid

The product of Example 15, of formula XIX (280 mg, 0.75 mmol) was stirred in a mixture of acetonitile (6 mL) and trimethylsilylbromide (TMSBr) (574 mg, 3.75 mmol) overnight at room temperature. The solvent was evaporated and the crude product was purified by chromatography on silica gel using dichloromethane:methanol:water (65:35:6) giving 188 mg (91%).

¹H NMR (D₂O) 1.65 (s, 6H), 2.02 (m,2H), 2.42 (s,3H), 3.40 (m, 2H), 4.24 (s, 2H), 5.12 (s, 2H), 8.11 (s, 1H).

³¹P NMR (H-decoupled, D₂O): 18.90 (s).

This structure can be represented by formula:

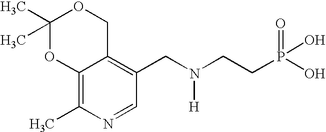

### Example 17

### Synthesis of (3-hydroxy-4-hydroxymethyl-2-methyl-5-pyridyl)-3-azabutyiphosphonic acid

The product of Example 16, of formula XX (168 mg, 0.53 mmol) was dissolved in acetic acid (80% in water, 10 mL) and heated to 60° C. for 5 hours. The solvent was removed by evaporation using toluene to codistill the water. The crude product was purified by chromatography on C-18 reverse phase silica gel using methanol:water (4:1) as eluent to give 57 mg (39%).

¹H NMR (D₂O) 2.05 (m, 2H), 2.52 (s, 3H), 3.38 (m, 2H), 4.42 (s, 2H), 4.96 (s, 2H), 7.87(s, 1H).

³¹P NMR (H-decoupled, D₂O): 18.90 (s).

This structure can be represented by formula:

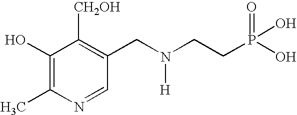

### Example 18

### Synthesis of diethyl (α⁴,3-O-isopropylidene-3-hydroxy-4-hydroxymethyl-2-methyl-5-pyridyl-2-hydroxyethylphosphonate

To a solution of diethyl methyl phosphite (0.29 mL, 2 mmol) in THF (20 mL) was added BuLi (2.5 M in hexane, 0.88 mL, 2.2 mmol), followed by (α⁴,3-O-isopropylidene-3-hydroxy-4-hydroxymethyl-2-methyl-5-pyridyl)methanal (Kortynk et al., J. Org. Chem., 29, 574-579 (1964)) (414 mg, 2 mmol) and the reaction mixture stirred at −78° C. for two hours. The solution was evaporated, dissolved in dichloromethane (50 mL), washed with saturated, aqueous NaHCO₃, dried (MgSO₄), evaporated and purified by chromatography on silica gel using ethyl acetate:hexane (1:2) as eluent to give 625 mg (87%).

¹H NMR(CDCl₃, TMS) 1.33 (m, 6H), 1.54 (s, 6H), 2.20 (m, 2H), 2.38 (s, 3H), 4.12 (m, 4H), 4.94 (s, 2H), 4.94 (s, 2H), 5.04 (t, 1H), 8.02 (s, 1H).

³¹P NMR (H-decoupled, CDCl₃): 29.03 (s).

This structure can be represented by formula:

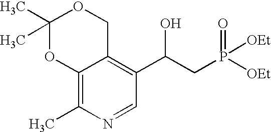

### Example 19

### Synthesis of diethyl (α⁴,3-O-isopropylidene-3-hydroxy-4-hydroxymethyl-2-methyl-5-pyridyl)-2acetoxyethylphosphonate

The product of Example 18, of structure XXII (300 mg, 0.84 mmol) was acetylated in pyridine (0.5 mL) and acetic anhydride (0.25 mL) at 0° C. for 5 minutes followed by 3 hours at room temperature. The solvent was removed by evaporation using toluene to codistill the solvents and the crude product was dissolved in dichloromethane (10 mL). This was washed with dilute HCl (10%, 5 mL), then saturated, aqueous NaHCO₃, dried (MgSO₄) and evaporated. Chromatography on silica gel using ethyl acetate:hexane (1:1) gave 258 mg (71%).

¹H NMR(CDCl₃, TMS) 1.21 (m, 6H), 1.54 (s, 6H), 2.03 (s,3H), 3.97 (m, 4H), 5.07 (dd, 2H), 5.83 (dd, 1H), 8.02 (s, 1H).

³¹P NMR (H-decoupled, CDCl₃): 25.01 (s).

This structure can be represented by formula:

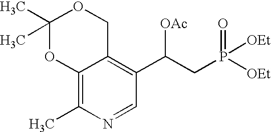

### Example 20

### Synthesis of diethyl (α⁴,3-O-isopropylidene-3-hydroxy-4-hydroxymethyl-2-methyl-5-pyridyl)-2-hydroxy-1,1-difluoroethylphosphonate

To a solution of lithiumdiisopropylamide (LDA) (2.0 M, 1 mL, 2 mmol) in THF (5 mL) was added BuLi (0.5 M, 0.2 mL, 0.1 mmol). The mixture was cooled to −40° C. followed by the addition of diethyl difluoromethyl phosphonate (0.32 mL, 2 mmol) and the reaction mixture stirred at this temperature for 30 minutes. The solution was cooled to −78° C. and (α⁴,3-O-Isopropylidene-3-hydroxy-4-hydroxymethyl-2-methyl-5-pyridyl)methanal (Kortynk et al., J. Org. Chem., 29, 574-579 (1964)) (414 mg, 2 mmol) added in THF (2 mL). The solution was allowed to come to room temperature and stirred overnight. The solvent was evaporated, the residue dissolved in dichloromethane (20 mL), washed with saturated, aqueous NaHCO₃, dried (MgSO₄), and evaporated. Purification by chromatography on silica gel using ethyl acetate:hexane (2:1) gave 528 mg (67%)

¹H NMR (CDCl₃, TMS) 1.35 (t, 3H), 1.38 (t, 3H), 1.52 (s, 3H), 1.55 (s, 3H), 2.39 (s,3H), 4.29 (m, 4H), 4.96 (dd, 3H), 8.09 (s, 1H).

¹⁹F NMR (CDCl₃)-125.99 (ddd), −114.55 (ddd).

³¹P NMR (H-decoupled, CDCl₃): 7.22 (dd).

This structure can be represented by formula:

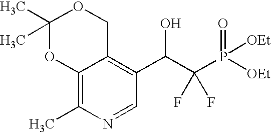

### Example 21

### Synthesis of diethyl (α⁴,3-O-isopropylidene-3-hydroxy-4-hydroxymethyl-2-methyl-5-pyridyl)-2-oxo-1,1-difluoroethylphosphonate

The product of Example 20, of structure XXIV, (420 mg, 1.06 mmol) was dissolved in toluene (50 mL) and MnO₂ (651 mg, 636 mmol) added. The mixture was heated to 50° C. and stirred overnight. The solution was cooled, filtered (Celite) and the solvent evaporated to give the crude product. Purification by chromatography on silica gel ethyl acetate (1:2) gave 201 mg (48%).

¹H nmr (CDCl₃, TMS) 1.39 (q, 6H), 1.56 (d, 6H), 2.51 (s,3H), 4.34 (m, 4H), 5.08 (s, 2H), 8.88 (s, 1H).

¹⁹F NMR (CDCl₃) −109.86 (d).

³¹P NMR (H-decoupled, CDCl₃): 3.96 (t).

This structure can be represented by formula:

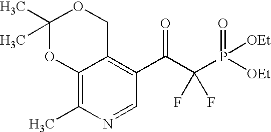

### Example 22

### Synthesis of diethyl (3-hydroxy-4-hydroxymethyl-2-methyl-5-pyridyl)-2-hydroxy-1,1-difluoroethylphosphonate

The product of Example 20, of structure XXIV (489 mg, 1.26 mmol) was dissolved in acetic acid (80% in water, 20 mL) and heated at 80° C. for 6 hours. The solvent was removed by evaporation by codistilling with toluene to remove last traces of acetic acid. The crude product was purified by chromatography on silica gel using dichloromethane:methanol:hexane (5:1:5) as eluent to give 171 mg (38%).

¹H NMR (CD₃OD) 1.32 (t, 3H), 1.37 (t, 3H), 2.43 (s,3H), 4.30 (m, 4H), 4.93 (dd, 2H), 5.39 (m, 2H), 8.07 (s, 1H).

¹⁹F NMR (CD₃OD) −125.55 (dd), −115.77 (dd).

³¹P NMR (H-decoupled, MeOD): 7.82 (dd).

This structure can be represented by formula:

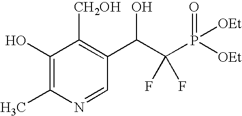

### Example 23

### Synthesis of diethyl (3-hydroxy-4-hydroxymethyl-2-methyl-5 pyridyl)-2-oxo-1,1-difluoroethylphosphonate

The product of Example 21, of structure XXV (198 mg, 0.51 mmol) was dissolved in acetic acid (80% in water, 20 mL) and heated at 80° C. for 6 hours. The solvent was removed by evaporation by codistilling with toluene to remove last traces of acetic acid. The crude product was purified by chromatography on silica gel using dichloromethane:methanol:hexane (5:1:5) as eluent to give 25 mg (14%).

¹H NMR (CDCl₃, TMS) 1.38 (m, 6H), 2.37 (s,3H), 4.33 (m, 4H), 4.92 (s, 1H), 7.88 (s, 1H).

¹⁹F (CDCl₃) −118.32 (d).

³¹P NMR (H-decoupled, CDCl₃): 5.90 (t).

This structure can be represented by formula:

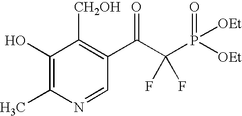

### Example 24

### Synthesis of diethyl (α⁴,3-O-isopropylidene-2-methyl-3-hydroxy-4-hydroxymethyl-5-pyridylmethyl)malonate

To a solution of diethyl malonate (0.76 mL, 798 mg, 4.98 mmol) in tetrahydrofuran (THF) (5 mL) was added LDA (5 M, 1 mL, 5.0 mmol) and stirred at 0° C. for 5 minutes. (α⁴,3-O-isopropylidene-3-hydroxy-4-hydroxymethyl-2-methyl-5-pyridyl)methylbromide (Imperalli et al, J. Org. Chem., 60, 1891-1894 (1995)) (1.36 g, 5.0 mmol) in THF (5 mL) was added. The reaction was stirred for 2 hours at 0° C. The solvent was evaporated and the residue was dissolved in Et₂O. This was washed with water, dried (MgSO₄) and evaporated to give the crude product. Purification of the crude mixture by chromatography on silica gel column using diethyl ether:hexane (1:1) gave the malonate derivative 769 mg (44%).

¹H NMR (CDCl₃, TMS) 1.23 (t, 6H), 1.54 (s, 6H), 2.37 (s, 3H), 3.04 (d, 2H), 3.63 (t, 1H), 4.18 (q, 4H), 4.86 (s, 2H), 7.87 (s, 1H).

### Example 25

### Level of expression of IL-6 in cells treated with PLP

Subcultured H9C2 cells (rat myocardium) (ATCC No. CRL-1446, from the American Type Culture Collection in Manassas, Va.) were plated into six well plates at a concentration of about 10⁶ cells per well and allowed to grow overnight. The wells were then treated with concentrations of pyridoxal-5′-phosphate at concentrations of about 0, 50, 100, 250, 500, and 1000 nM in the medium. The cells were incubated for about 40 minutes.

An oxidative stressor, 1 mM H₂O₂ (Sigma, St. Louis, Mo.), was then added to each well. Supernantant samples were collected from the wells before addition of the 1 mM H₂O₂, and after 2, 4, 6, and 12 hours of exposure. The supernatant samples were stored at −20° C. until analyzed. The levels of IL-6 were then analyzed in each of the samples using test kits from Biosource Intl. (Camarillo, Calif.). A control experiment established that pyridoxal-5′-phosphate did not interfere with the IL-6 detection systems of the test kits (data not shown).

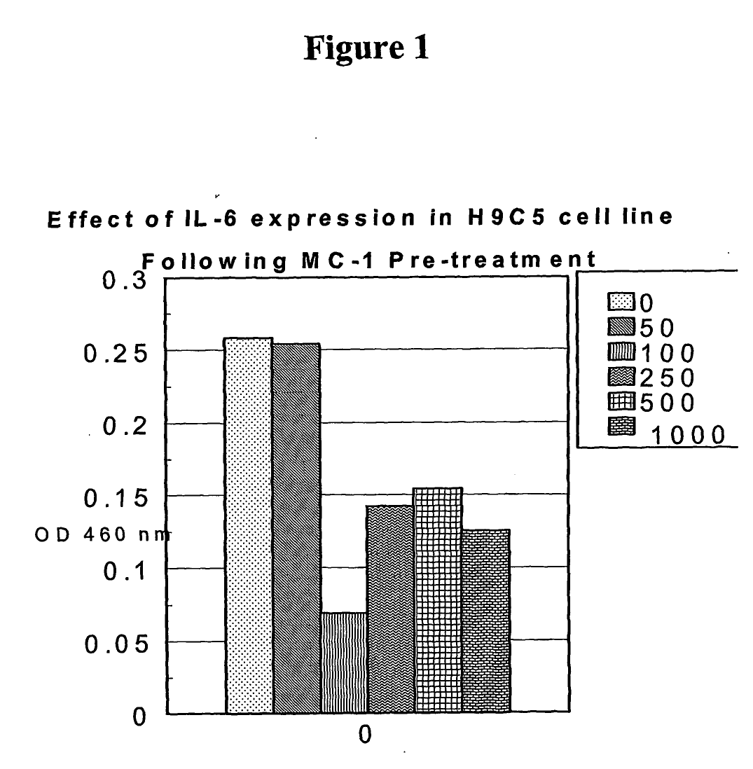

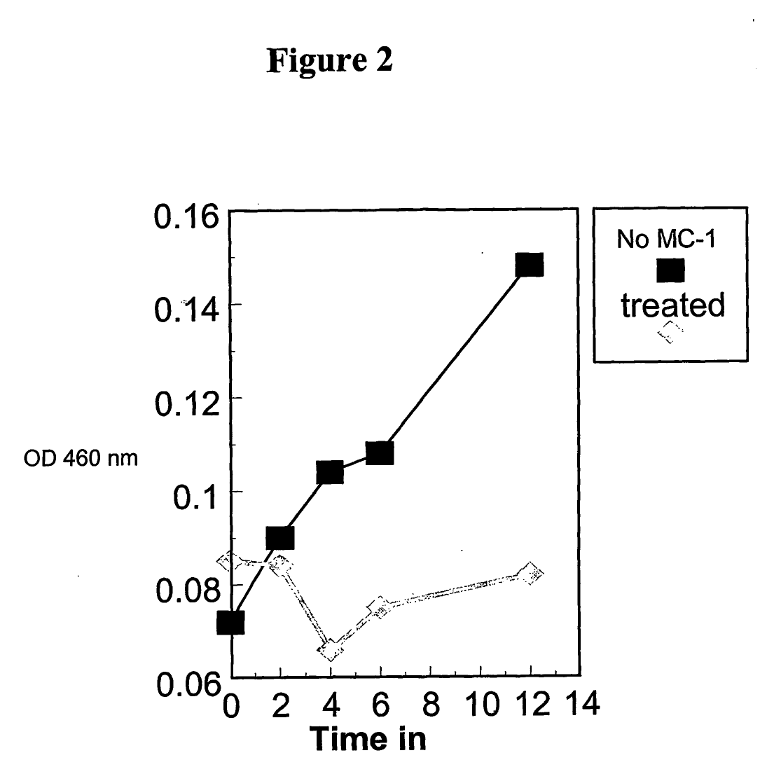

As shown in FIG. 1, IL-6 levels were dramatically increased after oxidative stress was applied. However, the cells that were pretreated with 100 nM or more of pyridoxal-5′-phosphate had decreased levels of IL-6 expression (FIG. 1). FIG. 2 shows that in the sample treated with 100 nM pyridoxal-5′-phosphate, this effect lasted for at least 12 hours. Pyridoxal-5′-phosphate did not effect the level of activated p38 (data not shown).

Because cytokines and IL-6 in particular play an important role in inflammation and inflammation involves modulation of cell death, this study demonstrates that pyridoxal-5′-phosphate can to be used as a modulator of cell death.

### Example 26

### Assays for Monitoring Modulation of Cell Death

The ability of compounds including, but not limited to pyridoxal-5′-phospate, pyridoxamine, pyridoxic acid, and compounds of formula V, VI, VIII, IX, or X to increase cellular viability and the cellular survival rate after a nominally fatal level of cellular stress can be assessed.

Cellular stresses used may include hydrogen peroxide to produce oxidative stress, Fas-signalling and TNF-alpha treatment to mediate cell death through death receptors, hypoxia, calpain activation, IL-8 treatment (inflammatory signal), or C5a (a member of the complement pathway) treatment.

A series of assays can be conducted to ascertain if cell death has been prevented. Some of the assays for cell viability may include trypan blue exclusion assay, the MTT assay, and clonogenicity assays. An Annexin V assay may help to determine if apoptosis, necrosis or a mixture thereof is being prevented. Some of the hallmarks of apoptosis may also be assayed with or without treatment by the compounds. Other potential assays include assays for specific apoptosis enzymes including caspase-3 assay or APAF-1 release, assays for DNA fragmentation including DNA laddering assays in agarose gels, acridine-orange staining, Tunnel staining for DNA ends, and assays for morphological staining including Wright-Giemsa staining. Other assays may include assays to determine if there are modifications to pathways known to be involved in cell death and survival, such as p38, JAK/STATS, and JNK.

Necrotic cell death assays may also be utilized. Assays utilized may include measuring STAT production, detection of cellular or mitochondrial swelling via microscopy, release of inflammatory cytokines like IL-6, or release of LDH.

Inflammation may also be investigated through the presence of C-reactive protein and IL-1.

Although embodiments of the invention have been described above, it is not limited thereto, and it will be apparent to persons skilled in the art that numerous modifications and variations form part of the present invention insofar as they do not depart from the spirit, nature and scope of the claimed and described invention.

We claim:

1. A method of modulating cell death comprising administering a therapeutically effective amount of at least one of pyridoxal-5′-phosphate, pyridoxic acid, pyridoxal, pyridoxine, or pyridoxamine.

2. A method of modulating cell death comprising administering a therapeutically effective amount of at least one compound of the formula 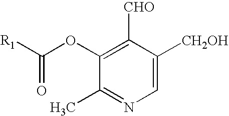
wherein
R₁ is alkyl or alkenyl, in which alkyl or alkenyl can be interrupted by nitrogen, oxygen, or sulfur, and can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy;
aryl, aryloxy, arylthio, or aralkyl,in which aryl can be substituted by alkyl, alkoxy, amino, hydroxy,halo, nitro, or alkanoyloxy; or
a pharmaceutically acceptable salt thereof.

3. The method of claim 2, wherein said R₁ is phenyl or naphthyl in which phenyl or naphthyl is unsubstituted or substituted by one or more groups of C₁₋₄ alkyl, C₁₋₄ alkoxy, amino, hydroxy, halo, nitro, or C₁₋₄ alkanoyloxy.

4. The method of claim 2, wherein said R₁ is (2-acetoxy-2-methyl)propanyl, dimethylamino, or 1-ethanoyloxy-1-methylethyl.

5. The method of claim 2, wherein said R₁ is tert-butyl.

6. The method of claim 2, wherein said R₁ is methoxy or ethoxy.

7. The method of claim 2, wherein said R₁ is toluyl, naphthyl, phenyl, acetylphenyl, or 1-ethanoyloxyphenyl.

8. The method of claim 2, wherein said R₁ is acetylsalicyl, dimethylamino, or 2,2-dimethylethyl.

9. The method of claim 2, wherein said compound is 2-methyl-3-toluoyloxy-4-formyl-5-hydroxymethylpyridine.

10. The method of claim 2, wherein said compound is 2-methyl-3-β-naphthoyloxy-4-formyl-5-hydroxymethylpyridine.

11. A method of modulating cell death comprising administering a therapeutically effective amount of at least one compound of the formula 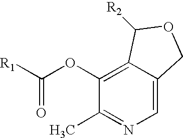
wherein
R₁ is alkyl or alkenyl, in which alkyl or alkenyl can be interrupted by nitrogen, oxygen, or sulfur, and can be substituted at the terminal carbon by hydroxy, alkoxy, alkanoyloxy, alkanoyloxyaryl, alkoxyalkanoyl alkoxycarbonyl, or dialkylcarbamoyloxy,
alkoxy,
dialkylamino;
alkanoyloxy,
alkanoyloxyaryl;
alkoxyalkanoyl;
alkoxycarbonyl;
dialkylcarbamoyloxy;
aryl, aryloxy, arylthio, or aralkyl,in which aryl can be substituted by alkyl, alkoxy, amino, hydroxy,halo, nitro, or alkanoyloxy, and
R₂ is a secondary amino group; or
a pharmaceutically acceptable salt thereof.

12. The method of claim 11, wherein said R₁ is phenyl or naphthyl in which phenyl or naphthyl is unsubstituted or substituted by one or more groups of C₁₋₄ alkyl, C₁₋₄ alkoxy, amino, hydroxy, halo, nitro, or C₁₋₄ alkanoyloxy.

13. The method of claim 11, wherein said R₁ is (2-acetoxy-2-methyl)propanyl, dimethylamino, or 1-ethanoyloxy-1-methylethyl.

14. The method of claim 11, wherein said wherein R₁ is tert-butyl.

15. The method of claim 11, wherein said wherein R₁ is methoxy or ethoxy.

16. The method of claim 11, wherein said R₁ is toluyl, naphthyl, phenyl, or 1-ethanoyloxyphenyl.

17. The method of claim 11, wherein said R₁ is dimethylamino, acetylsalicyl, or 2,2-dimethylethyl.

18. The method of claim 11, wherein said R₂ is a group of the formula 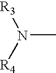
wherein R₃ and R₄ are each independently alkyl or when taken together form a ring with the nitrogen atom and which ring may optionally be interrupted by a nitrogen or oxygen atom.

19. The method of claim 11, wherein said R₂ is piperidino.

20. The method of claim 11, wherein said R₂ is morpholino or piperazino.

21. The method of claim 11, wherein said compound is 1-morpholino-1,3-dihydro-7-(p-toluoyloxy)-6-methylfuro(3,4-c)pyridine.

22. The method of claim 11, wherein said compound is 1-morpholino-1,3-dihydro-7-(β-naphthoyloxy)-6-methylfuro(3,4-c)pyridine.

23. The method of claim 11, wherein said compound is 1-morpholino-1,3-dihydro-7-pivaloyloxy-6-methylfuro(3,4-c)pyridine.

24. The method of claim 11, wherein said compound is 1-morpholino-1,3-dihydro-7-(dimethylcarbamoyloxy-6-methylfuro(3,4-c)pyridine.

25. The method of claim 11, wherein said compound is 1-morpholino-1,3-dihydro-7-acetylsalicyloxy-6-methylfuro(3,4-c)pyridine.

26. A method of modulating cell death comprising administering a therapeutically effective amount of at least one compound of the formula 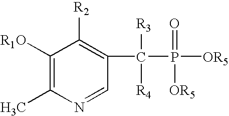
wherein
R₁ is hydrogen or alkyl;
R₂ is —CHO, —CH₂OH, —CH₃, —CO₂R₆ in which R₆ is hydrogen, alkyl, or aryl;
or
R₂ is —CH₂—O-alkyl- in which alkyl is covalently bonded to the oxygen at the 3-position instead of R₁;
R₃ is hydrogen and R₄ is hydroxy, halo, alkoxy, alkanoyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable salt thereof.

27. The method of claim 26, wherein said R₁ is hydrogen.

28. The method of claim 26, wherein said R₂ is —CH₂OH, or —CH₂—O-alkyl- in which alkyl is covalently bonded to the oxygen at the 3-position instead of R₁.

29. The method of claim 26, wherein said R₃ is hydrogen and R₄ is F, MeO—, or CH₃C(O)O—.

30. The method of claim 26, wherein said R₃ and R₄ are F.

31. The method of claim 26, wherein said R₅ is alkyl or aralkyl.

32. The method of claim 26, wherein said R₅ is t-butyl or benzyl.

33. A method of claim 26, wherein said compound is

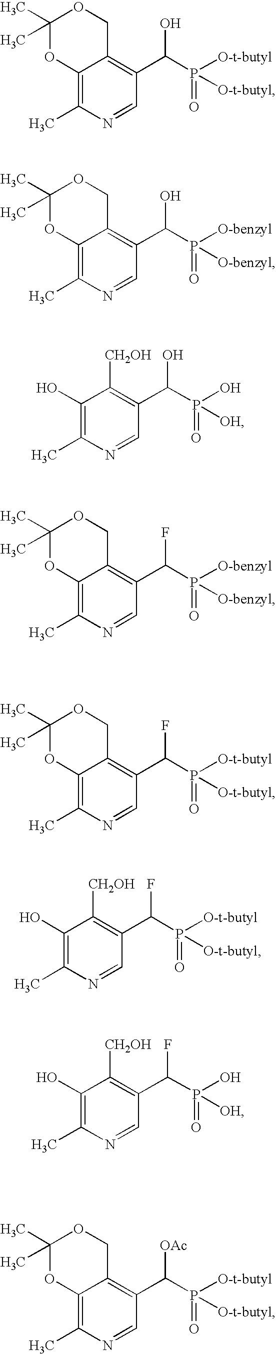

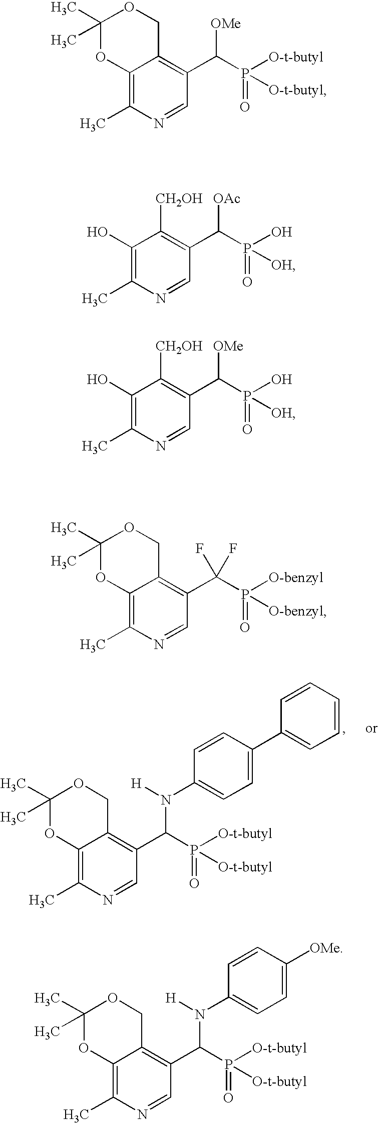

34. A method modulating cell death comprising administering a therapeutically effective amount of at least one compound of the formula 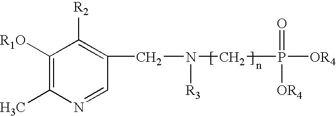
wherein
R₁ is hydrogen or alkyl;
R₂ is —CHO, —CH₂OH, —CH₃ or CO₂R₅ in which R₅ is hydrogen, alkyl, or aryl; or
R₂ is —CH₂O-alkyl- (in which alkyl is covalently bonded to the oxygen at the 3-position instead of R₁);
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl; and
n is 1 to 6;
or a pharmaceutically acceptable salt thereof

35. The method of claim 34, wherein said R₁ is hydrogen.

36. The method of claim 34, wherein said R₂ is —CH₂OH, or —CH₂—O-alkyl- in which alkyl is covalently bonded to the oxygen at the 3-position instead of R₁.

37. The method of claim 34, wherein said R₃ is hydrogen.

38. The method of claim 34, wherein said R₄ is alkyl or H.

39. The method of claim 34, wherein said R₄ is ethyl.

40. The method of claim 34, wherein said compound is

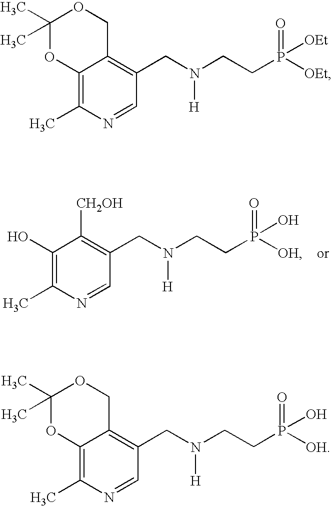

41. A method modulating cell death comprising administering a therapeutically effective amount of at least one compound of the formula 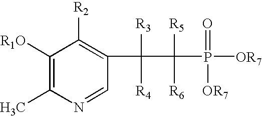
in which
R₁ is hydrogen or alkyl;
R₂ is —CHO, —CH₂OH, —CH₃ or —CO₂R ₈ in which R₈ is hydrogen, alkyl, or aryl; or
R₂ is —CH_{2—}O-alkyl- in which alkyl is covalently bonded to the oxygen at the 3-position instead of R₁;
R₃ is hydrogen and R₄ is hydroxy, halo, alkoxy or alkanoyloxy; or
R₃ and R₄ can be taken together to form ═O;
R₅ and R₆ are hydrogen; or
R₅ and R₆ are halo; and
R₇ is hydrogen, alkyl, aryl, aralkyl, or —CO₂R₈ in which R₈ is hydrogen, alkyl, aryl, or aralkyl;
or a pharmaceutically acceptable salt thereof.

42. The method of claim 41, wherein R₁ is hydrogen.

43. The method of claim 41, wherein R₂ is —CH₂O or —CH₂—O-alkyl- in which alkyl is covalently bonded to the oxygen at the 3-position instead of R₁.

44. The method of claim 41, wherein said R₄ is —OH or CH₃C(O)O—.

45. The method of claim 41, wherein said R₃ and R₄ taken together form ═O.

46. The method of claim 41, wherein said R₅ and R₆ are F.

47. The method of claim 41, wherein said R₇ is alkyl.

48. The method of claim 41, wherein said R₇ is ethyl.

49. The method of claim 41, wherein said compound is [chemical structure]
